(19) United States
(12) Patent Application Publication
(10) Pub. No.: US 2020/0305714 A1
(43) Pub. Date: Oct. 1, 2020

(54) IOT SYSTEM AND MONITORING METHOD FOR MONITORING ASSOCIATION BETWEEN INDOOR ENVIRONMENT AND HEALTH OF ELDERLY PEOPLE

(71) Applicant: Dalian University of Technology, Dalian (CN)

(72) Inventors: Yang Lv, Dalian (CN); Zhimeng Wang, Dalian (CN)

(21) Appl. No.: 16/656,606

(22) Filed: Oct. 18, 2019

(30) Foreign Application Priority Data
Mar. 29, 2019 (CN) 201910253201.5

(51) Int. Cl. A61B 5/00; G16H 50/30; G16H 40/67; H04L 12/28

(52) U.S. Cl. CPC A61B 5/0022; G16H 50/30; A61B 5/0024; A61B 5/0004; H04L 12/2827; G16H 40/67

(57) ABSTRACT

Disclosed are an IoT system and a monitoring method for monitoring association between indoor environment and health of elderly people. Such IoT system comprises an environmental and health parameter monitoring system and an IoT data association platform, the monitoring system performs data transmission with the IoT data association platform by a GPRS wireless communication module and a data receiving API interface. The monitoring system is combined with the IoT data association platform. The IoT system has a small physical volume and can be fixed to the periphery of the human body by wearing. The constructed IoT environment and health association data platform can remotely and online monitor change of the temperature and humidity and particle concentration in the environment where elderly people are located, and remotely and online monitor the blood pressure and heart rate of elderly people. The health risks can be predicted in real time by input environmental data.

### CROSS REFERENCE TO RELATED APPLICATIONS

The present application claims the benefit of Chinese Patent Application No. 201910253201.5 filed on Mar. 29, 2019, the contents of which are incorporated herein by reference in their entirety.

### FIELD OF THE INVENTION

The present invention relates to an IoT (Internet of Things) system and a monitoring method for monitoring association between indoor environment and health of elderly people, belonging to the field of environmental and health monitoring, information communication and data algorithm.

### DESCRIPTION OF THE RELATED ART

With the rapid development of modern social economy, the environmental regulation device such as air-conditioning and heating creates an indoor thermal environment that satisfies the comfort of the human body, but also creates a large temperature difference between an air-conditioning/heating room and a non-air-conditioning/heating room. When moving between the two environments, the health of the body may be considerably damaged due to the large temperature difference, for example it is easy to catch a cold, cause a cold, and the like. Especially for elderly people, it is easy to induce cardiovascular and cerebrovascular diseases, for example serious diseases such as stroke, myocardial infarction, and the like. Meantime, due to the increasingly prominent smog problem in recent years, the environment in which people live is also harmed by particle pollution. For elderly people, particle pollution, especially PM_{2.5} pollution, can easily increase the incidence of cardiovascular and cerebrovascular diseases in elderly people, and pose a great threat to the health of elderly people.

As China's population aging problem becomes more and more serious, the healthy living environment of elderly people should receive more attention. At present, most of the living environment monitoring technology for elderly people is fixed-point monitoring placed in the room. When elderly people leave the room, the data of the environmental monitoring point is difficult to reflect the current environmental status of the elderly people. In addition, there are some wearable monitoring devices that can monitor the air quality around the human body in real time, but there are few products that combine temperature and humidity with particle concentration monitoring and start from the perspective of healthy living environment of elderly people. The communication manners of such devices are mainly Bluetooth and WiFi. For most elderly people, especially those in rural areas, the operations of mobile phone Bluetooth connected wearable devices are particularly complicated and difficult to learn, and the network facilities in the home are lacked, so that such wearable monitoring devices are difficult to function.

On the other hand, due to the physique sensitivity of elderly people, the physiological condition of the body changes when subjected to changes in the surrounding air environment. When the changes in physiological indicators exceed the immune limit of elderly people, various diseases are caused, among which cardiovascular and cerebrovascular diseases are the main types of diseases. At present, device manufacturers on the market focus on their own products. When elderly users use devices produced by different manufacturers, the data between devices of different manufacturers often does not interwork with each other, which causes certain obstacles for an overall assessment and monitoring association between environment and health of elderly resident. Meanwhile, different manufacturers use different data platforms, which causes considerable difficulty in use for elderly users.

Patent No. CN201510698675.2 discloses a health management system based on IoT home. A management apparatus provides health management information by an interactive interface, generates a control signal according to the health management information, and controls the home appliance according to the control signal. However, the system lacks monitoring of the surrounding environment when the human body moves, and meantime the method of generating the control signal according to the health management information is based on the user's manual adjustment to achieve the adjustment effect, which lacks adaptability in different environments.

### SUMMARY OF THE INVENTION

In order to solve the problems existing in the prior art, the present invention provides an IoT system and a monitoring method for monitoring association between indoor environment and health of elderly people, which combine an environmental and health parameter wearable monitoring system with an IoT data association platform, utilize GPRS wireless communication technology to achieve real-time uploading of environmental and health monitoring data, and output and display health risk situations caused by the environment in real time.

The technical solution adopted by the present invention is an IoT system for monitoring association between environment and health of elderly people, comprises an environmental and health parameter monitoring system and an IoT data association platform, the environmental and health parameter monitoring system contains a power supply system, a air quality monitoring system, a physiological parameter monitoring system, a STM32 SCM and a GPRS wireless communication module; the IoT data association platform contains a data receiving API interface, a cloud server and a data visualization terminal, and the environmental and health parameter monitoring system performs data transmission with the IoT data association platform by the GPRS wireless communication module and the data receiving API interface.

The power supply system contains a power module, a USB charging port and a circuit switch, the air quality monitoring system is composed of a temperature and humidity sensor module and a particle sensor module, the physiological parameter monitoring system is composed of a heart rate sensor module and a blood pressure sensor module, the temperature and humidity sensor module achieves single-wire bidirectional data communication with the STM32 SCM by an IO port, the particle sensor module, the heart rate sensor module and the blood pressure sensor module respectively performs data transmission with the STM32 SCM by UART communication, and the cloud server is composed of a relational database and an environment and health association algorithm module.

A monitoring method of the IoT system for monitoring association between environment and health of elderly people comprises the steps of:

1) the circuit switch is closed, the circuit is connected, the STM32 SCM sends signals to wake up the temperature and humidity sensor module, the particle sensor module, the blood pressure sensor module and the heart rate sensor module respectively, and monitored temperature and humidity data, PM₁₀ and PM_{2.5} data, blood pressure data and heart rate data are transmitted to the STM32 SCM by data ports;
2) the STM32 SCM processes the received temperature, humidity, PM₁₀ and PM_{2.5}, blood pressure and heart rate data and transmits the processed data to the GPRS wireless communication module;
3) after receiving the data, the GPRS wireless communication module sends the data to the data receiving API interface by an antenna, and the data receiving API interface simultaneously receives data of different environmental and health monitoring devices from other IoT platforms;
4) the data receiving API interface transfers all the received data to the relational database, and the relational database stores all the data of each residence as a data set;
5) all the data stored in the relational database is calculated by the environment and health association algorithm, an environment and health association model suitable for each elderly user is obtained, a real-time environmental parameter is input to the model, the environment and health association model outputs and displays health risk situations caused by the environment in real time, and the elderly users browse all data related to themselves and health risk situations caused by their environment by a data visualization terminal.

The beneficial effects of the present invention are that: such IoT system that monitors association between environment and health of elderly people includes an environmental and health parameter monitoring system and an IoT data association platform, the environmental and health parameter monitoring system contains a power supply subsystem, a air quality monitoring subsystem, a physiological parameter monitoring subsystem, a STM32 SCM (Single Chip Micyoco) and a GPRS wireless communication module; the IoT data association platform contains a data receiving API interface, a cloud server and a data visualization terminal, and the environmental and health parameter monitoring system performs data transmission with the IoT data association platform by the GPRS wireless communication module and the data receiving API interface. The technical solution of the present invention combines the environmental and health parameter monitoring system with the IoT data association platform, and can monitor the impact of environmental factors in different places on the health of elderly people in real time. Meantime the IoT data association platform also provides other IoT environments and health monitoring devices with a unified communication interface, and integrates the environmental and health data monitored by different manufacturers and different devices by binding data such as user's residential address and elderly people related information. Elderly users can independently select a data type (for example data parameter such as temperature, relative humidity, carbon dioxide concentration, formaldehyde concentration, PM_{2.5} concentration, blood pressure, heart rate, and sleep) to be monitored according to their own situations and preferences. All data is uploaded to a relational database of the cloud server, providing each elderly user with an environmental and health association model that is consistent with their own health condition. The IoT system has a small physical volume and can be fixed to the periphery of the human body by wearing. The constructed IoT environment and health association data platform can remotely and online monitor change of the temperature and humidity and particle concentration in the environment where elderly people are located, and remotely and online monitor the blood pressure and heart rate of elderly people. The health risks can be predicted in real time by the input environmental data. Elderly people can browse all data related to themselves and the health risk situations caused by their environment by a data visualization client, without complicated operations.

### BRIEF DESCRIPTION OF THE DRAWINGS

FIG. 1 is a structure diagram of an environmental and health parameter monitoring system.

FIG. 2 is a circuit diagram of an environmental and health parameter monitoring system.

FIG. 3 is a structural diagram of an IoT data association platform.

FIG. 4 is a block diagram of the environment and health association algorithm.

### DETAILED DESCRIPTION OF THE EMBODIMENTS

The technical solutions of the present invention are clearly and completely described below in conjunction with the accompanying drawings and specific embodiments.

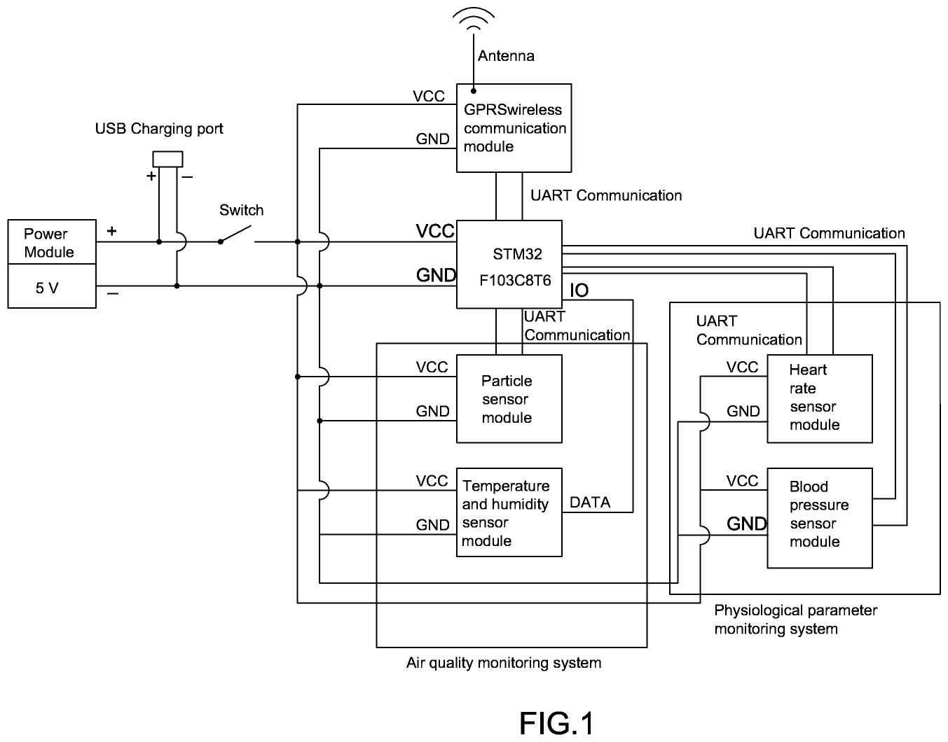

FIG. 1 shows a structure diagram of an environmental and health parameter monitoring system. Such IoT system that monitors association between environment and health of elderly people includes an environmental and health parameter monitoring system and an IoT data association platform, the environmental and health parameter monitoring system contains a power supply system, a air quality monitoring system, a physiological parameter monitoring system, a STM32 SCM and a GPRS wireless communication module; the IoT data association platform contains a data receiving API interface, a cloud server and a data visualization terminal, and the environmental and health parameter monitoring system performs data transmission with the IoT data association platform by the GPRS wireless communication module and the data receiving API interface.

The power supply system contains a power module, a USB charging port and a circuit switch. The air quality monitoring system is composed of a temperature and humidity sensor module and a particle sensor module. The physiological parameter monitoring system is composed of a heart rate sensor module and a blood pressure sensor module. The temperature and humidity sensor module achieves single-wire bidirectional data communication with the STM32 SCM by an IO port. The particle sensor module, the heart rate sensor module and the blood pressure sensor module respectively performs data transmission with the STM32 SCM by UART communication. The cloud server is composed of a relational database and an environment and health association algorithm module.

The monitoring method of such IoT system that monitors association between environment and health of elderly people includes the following steps:

1) The circuit switch is closed, the circuit is connected, the STM32 SCM sends signals to wake up the temperature and humidity sensor module, the particle sensor module, the blood pressure sensor module and the heart rate sensor module respectively, and monitored temperature and humidity data, PM₁₀ and PM_{2.5} data, blood pressure data and heart rate data are transmitted to the STM32 SCM by data ports;
2) The STM32 SCM processes the received temperature, humidity, PM₁₀ and PM_{2.5}, blood pressure and heart rate data and transmits the processed data to the GPRS wireless communication module;
3) After receiving the data, the GPRS wireless communication module sends the data to the data receiving API interface by an antenna, and the data receiving API interface simultaneously receives data of different environmental and health monitoring devices from other IoT platforms;
4) The data receiving API interface transfers all the received data to the relational database, and the relational database stores all the data of each residence as a data set;
5) All the data stored in the relational database is calculated by the environment and health association algorithm, an environment and health association model suitable for each elderly user is obtained, a real-time environmental parameter is input to the model, the environment and health association model outputs and displays health risk situations caused by the environment in real time, and the elderly users browse all data related to themselves and health risk situations caused by their environment by a data visualization terminal.

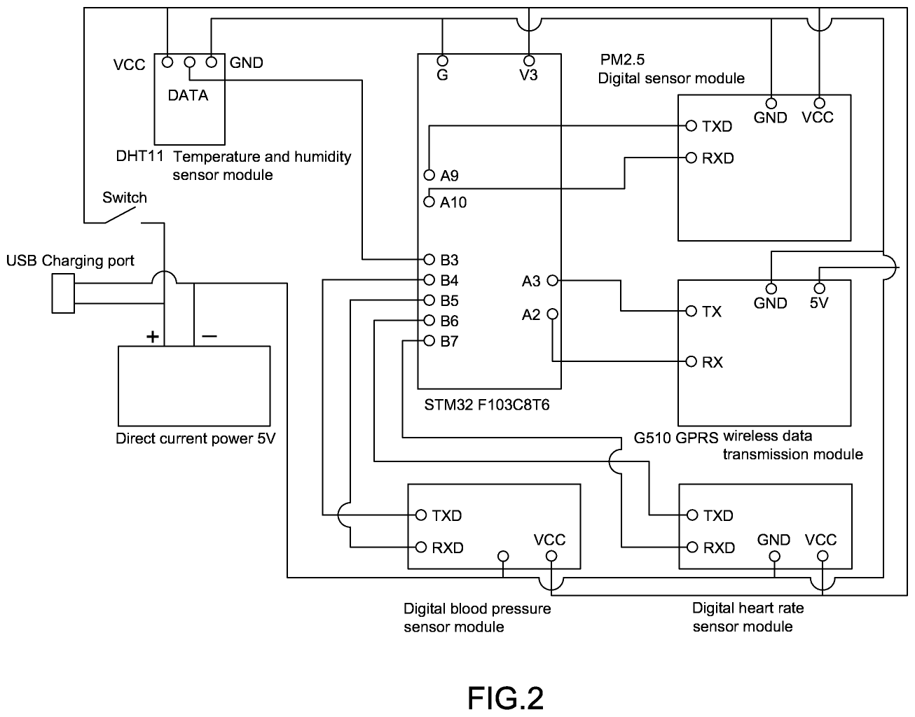

The specific working process of such IoT system that monitors association between environment and health of elderly people is: the circuit switch is closed, and the circuit is connected. On the one hand, the STM32 SCM sends a signal to wake up the temperature and humidity sensor module by a B3 data port (TO data transmission port), and the temperature and humidity sensor starts to work. The monitored temperature and relative humidity data are sent to the B3 data port of the STM32 SCM by a DATA data port. 40-bit data, including temperature, relative humidity, and checksum is transmitted each time. Meantime, the STM32 SCM wakes up the particle sensor module by UART communication, and the particle sensor starts to work. The monitored PM₁₀ and PM_{2.5} data are transmitted to the STM32 SCM by UART communication. That is, by an A9 data port and an A10 data port of the STM32 SCM, the data communication with a TXD data port and a RXD data port of the particle sensor is performed respectively. The concentration values of PM₁₀ and PM_{2.5} are transmitted by hexadecimal digital signals, and the data size of each transmission is 80 bits. On the other hand, the STM32 SCM wakes up the blood pressure sensor module and the heart rate sensor module by UART communication, and the blood pressure sensor module and the heart rate sensor module start to work. The monitored blood pressure and heart rate data are transmitted to the STM32 SCM by UART communication. That is, by a B4 data port and a B5 data port, the data communication with a TXD data port and a RXD data port of the blood pressure sensor is performed respectively, and a B6 data port and a B7 data port of the STM32 SCM perform data communication with a TXD data port and a RXD data port of the heart rate sensor respectively. Both the blood pressure (including diastolic pressure and systolic pressure) data and heart rate data are transmitted by hexadecimal digital signals. The blood pressure data size of each transmission is 80 bits, and the heart rate data size of each transmission is 40 bits (as shown in FIG. 2).

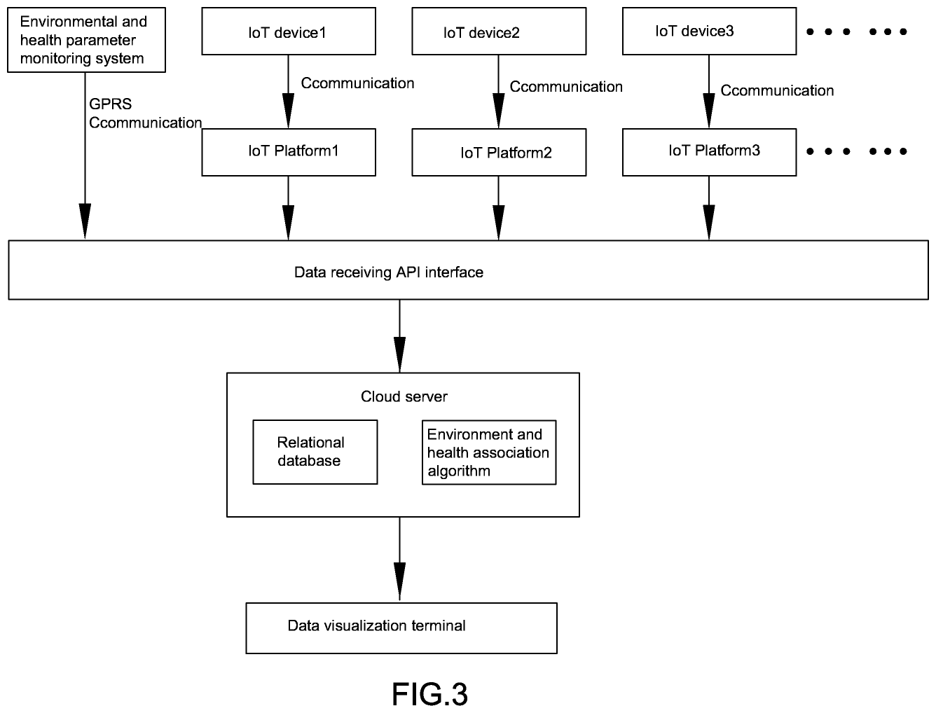

After receiving the data of temperature and humidity, particle, blood pressure and heart rate, the STM32 SCM processes the received data, converts a hexadecimal digital signal into a decimal character string and transmits it to the GPRS wireless communication module by UART communication. That is, an A2 data port and an A3 data port communicates with a TX data port and a RX data port of the G510 GPRS wireless communication module respectively. After receiving the data, the GPRS wireless communication module sends the data to the data receiving API interface by an antenna, and the data receiving API interface uploads the data to the IoT data association platform. Meantime, the IoT data association platform connects to other third-party IoT platforms by the data receiving API interface, and the data receiving API interface simultaneously collects data of various environmental and health monitoring devices from other IoT platforms. The data receiving API interface transfers all received data to a relational database, which stores data of each residence as a data set. An environment and health association algorithm module trains the data set in the relational database by a deep learning network to obtain an environment and health association model suitable for each resident. When a real-time environmental parameter is input to the model, health risk situations caused by the environment can be output and displayed in real time. The resulting health risk situation. Elderly users can browse all data related to themselves and health risk situations caused by their environment by a data visualization terminal, such as PC and mobile client (as shown in FIG. 3).

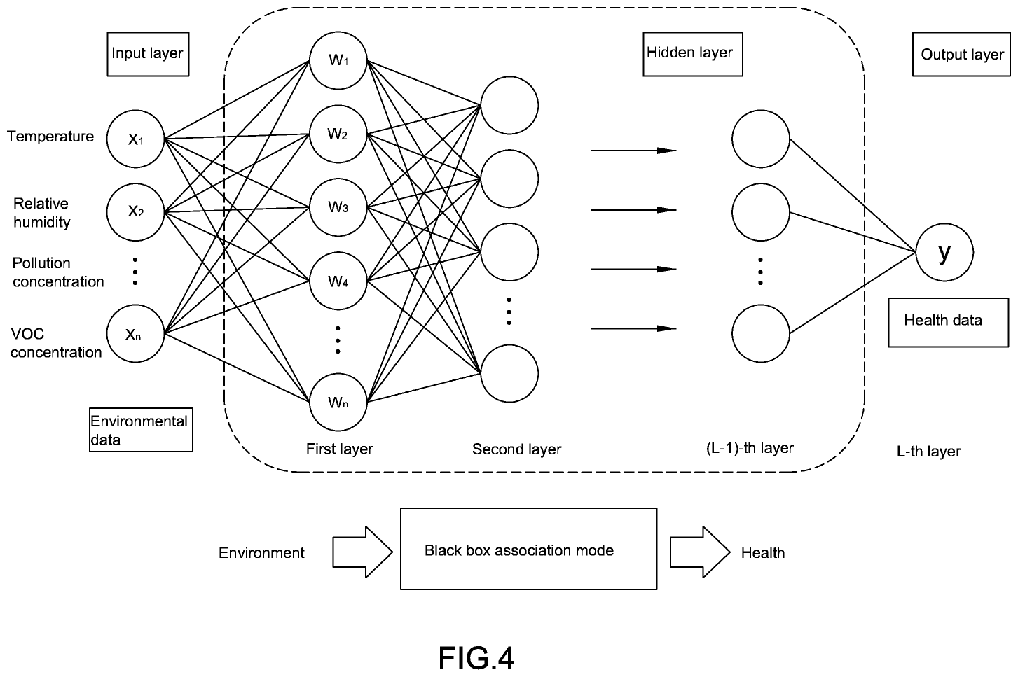

The environment and health association algorithm module trains a personalized deep learning network model only suitable for residence in which an elderly user lives and the elderly user's health parameter prediction, mainly by constructing a deep learning network and using daily environment and health data provided in the life process of the elderly user. In a certain time scale, the environmental parameter is selected as the input parameter x, and the health data is used as the output parameter y. After the calculation by the deep learning network mode, the predicted value y′=wx+b is obtained. The loss function and the cost function are constructed according to y′ and y. The weighting parameters w and b are updated by the gradient descent method. Finally the personalized environment and health association model is trained. Meantime, the daily data is re-used as training data, and the personalized deep learning network model is continuously improved. The model takes the environmental data related to elderly people as input, outputs the health data risk as the health prediction result of the environment, and displays the health risk situations caused by the environment in real time by the digital visualization terminal of the IoT data association platform (as shown in FIG. 4).

In summary, such IoT system for monitoring association between environment and health of elderly people has a small physical volume and can be fixed to the periphery of the human body only by wearing, such as worn in front of the chest or tied to the arm. The constructed IoT environment and health association data platform can remotely and online monitor change of the temperature and humidity and particle concentration in the environment where elderly people are located, and remotely and online monitor the blood pressure and heart rate of elderly people. The environmental and health association models can predict the health risks in real time by the environmental data. It is suitable for medical staff, family members, and elderly people themselves to meet the needs of healthy living of elderly people. When the number of users in a certain area increases to a certain extent, it can also provide basic scientific research and government related departments in the area with data-based decision basis.

What is claimed is:

1. An IoT system for monitoring association between environment and health of elderly people, characterized by comprising: an environmental and health parameter monitoring system and an IoT data association platform, the environmental and health parameter monitoring system contains a power supply system, a air quality monitoring system, a physiological parameter monitoring system, a STM32 SCM and a GPRS wireless communication module; the IoT data association platform contains a data receiving API interface, a cloud server and a data visualization terminal, and the environmental and health parameter monitoring system performs data transmission with the IoT data association platform by the GPRS wireless communication module and the data receiving API interface.

2. The IoT system for monitoring association between environment and health of elderly people according to claim 1, characterized in that the power supply system contains a power module, a USB charging port and a circuit switch, the air quality monitoring system is composed of a temperature and humidity sensor module and a particle sensor module, the physiological parameter monitoring system is composed of a heart rate sensor module and a blood pressure sensor module, the temperature and humidity sensor module achieves single-wire bidirectional data communication with the STM32 SCM by an IO port, the particle sensor module, the heart rate sensor module and the blood pressure sensor module respectively performs data transmission with the STM32 SCM by UART communication, and the cloud server is composed of a relational database and an environment and health association algorithm module.

3. A monitoring method of the IoT system for monitoring association between environment and health of elderly people according to claim 1, characterized by comprising the steps of:
1) the circuit switch is closed, the circuit is connected, the STM32 SCM sends signals to wake up the temperature and humidity sensor module, the particle sensor module, the blood pressure sensor module and the heart rate sensor module respectively, and monitored temperature and humidity data, PM₁₀ and PM_{2.5} data, blood pressure data and heart rate data are transmitted to the STM32 SCM by data ports;
2) the STM32 SCM processes the received temperature, humidity, PM₁₀ and PM_{2.5}, blood pressure and heart rate data and transmits the processed data to the GPRS wireless communication module;
3) after receiving the data, the GPRS wireless communication module sends the data to the data receiving API interface by an antenna, and the data receiving API interface simultaneously receives data of different environmental and health monitoring devices from other IoT platforms;
4) the data receiving API interface transfers all the received data to the relational database, and the relational database stores all the data of each residence as a data set;
5) all the data stored in the relational database is calculated by the environment and health association algorithm, an environment and health association model suitable for each elderly user is obtained, a real-time environmental parameter is input to the model, the environment and health association model outputs and displays health risk situations caused by the environment in real time, and the elderly users browse all data related to themselves and health risk situations caused by their environment by a data visualization terminal.
